(19) United States
(12) Patent Application Publication
(10) Pub. No.: US 2018/0185490 A1
(43) Pub. Date: Jul. 5, 2018

(54) ANTIFUNGAL PREPARATIONS INTENDED FOR THE TOPICAL TREATMENT OF ONYCHOMYCOSIS

(71) Applicant: R&D PHARMA, Monaco (MC)

(72) Inventor: Pascal VIANT, Monaco (MC)

(73) Assignee: R&D PHARMA, Monaco (MC)

(21) Appl. No.: 15/399,586

(22) Filed: Jan. 5, 2017

(51) Int. Cl. A61K 47/22; A61K 31/343; A61K 31/4412; A61K 31/5375; A61K 31/135; A61K 47/12

(52) U.S. Cl. CPC A61K 47/22; A61K 31/343; A61K 47/12; A61K 31/5375; A61K 31/135; A61K 31/4412

(57) ABSTRACT

Antifungal preparations in solution in gamma-butyrolactone, useful for the topical treatment of onychomycosis, whose therapeutic effect may be reinforced by the keratolytic action of the salicylic acid and the preparations may advantageously be made viscous, gelled or filmogenic, with or without a colorant.

### BACKGROUND OF THE INVENTION

### 1. Field of the Invention

Treating fungal infections of the nail (onychomycosis) is both long and fastidious. Because of the slow growth of the nail, the treatment has to be applied for approximately six months for the nails of the hand and twelve months for the nails of the foot.

### 2. Description of the Related Art

The treatment may be topical or systemic (by mouth). If the latter seems simpler for the patient, systemic treatment (by mouth) alone is insufficient. Indeed, on the one hand, it is insufficiently effective, on the other, the long duration of the treatment increases the risk of potentially undesirable side effects appearing, with regard to which biological monitoring is necessary.

Topical treatment alone, or one associated with systemic treatment (where the matrix is affected) is often the rule, in parallel with the avulsion of the diseased part of the nail and careful hygiene of the hands and/or feet.

Among the antifungal treatments used, the following may be mentioned: griseofulvin, imidazole derivatives (bifonazole), ciclopirox, ciclopirox olamine, amorolfine or terbinafine.

Because of its specific action on dermatophytes (Microsporum, Trichophyton, Epidermophyton), griseofulvin remains a valid therapeutic agent in the treatment of onychomycosis. Clinical practice has shown no appearance of resistant strains.

However, the use of griseofulvin in the topical treatment of onychomycosis has proved impossible up to now because of its inactivity using this method due to its insolubility and its inability to sufficiently penetrate the diseased areas of the nails.

### SUMMARY OF THE INVENTION

The aim of the present invention is to propose preparation enabling the effective elimination of mycosis.

The invention thus relates to antifungal preparations, wherein they are solutions comprising gamma-Butyrolactone as the solvent vehicle.

According to one characteristic of the invention, the preparations are composed of between 1 and 6% by weight of salicylic acid.

According to another characteristic of the invention, the preparations are composed both of between 1 and 6% by weight of salicylic acid and between 1 and 6% by weight of benzoic acid.

According to yet another characteristic of the invention, the preparations are composed of between 0.5 and 7% by weight of griseofulvin.

According to yet another characteristic of the invention, the preparations are composed of between 0.5 and 5% by weight of an antifungal from the group of imidazoles (e.g. bifonazole).

According to yet another characteristic of the invention, the preparations are composed of between 0.5 and 10% by weight of ciclopirox or ciclopirox olamine.

According to yet another characteristic of the invention, the preparations are composed of between 0.5 and 10% by weight of amorolfine.

According to yet another characteristic of the invention, the preparations are composed of between 0.5 and 5% by weight of terbinafine.

According to yet another characteristic of the invention, the preparations are composed in addition of between 5 and 50% by weight of one or several biologically acceptable solvents, such as ethanol, propylene glycol, acetone, ethyl acetate, butyl acetate or dimethylsulphoxide.

According to yet another characteristic of the invention, the preparations are composed of between 1 and 10%, preferentially between 1.5 and 5% by weight, of an agent, such as colloidal silica, enabling a viscous or gelled solution to be obtained.

According to yet another characteristic of the invention, the preparations are made filmogenic using appropriate agents, such as acrylate copolymer and methyl acrylate (EUDRAGIT) or copolymer of methyl-vinyl-ether and maleic acid monobutyl ester, incorporated in proportions of between 0.5 and 10% by weight.

According to another characteristic of the invention, the preparations are composed of an emulsifying and detergent agent, such as polyoxyethylenic alcohols (e.g. Cetomacrogol 1000) or Polysorbate 80.

According to yet another characteristic of the invention, the preparations may be tinted using mineral, organic, or more advantageously vegetal colorants.

According to yet another characteristic of the invention, the preparations can be used for the topical treatment of onychomycosis.

### DETAILED DESCRIPTION OF PREFERRED EMBODIMENTS

The present invention relates to the observation of the remarkable property of gamma-butyrolactone (GBL) as a solvent for griseofulvin; it is possible for solutions of the latter to be obtained at 7% (weight/weight).

It also acts as a solvent for other antifungals:

bifonazole, ciclopirox, ciclopirox olamine, amorolfine or terbinafine. Solutions at between 0.5 and 10% of these different antifungals in GBL are advantageously prepared.

Solubilisation tests for griseofulvin or other antifungals in N-methylpyvolidone (NMP) have been described previously (STOUGHTON, 1975). However, NMP suffers the disadvantage of being of those solvents whose pharmacopoeia limits their content (class 2) in active ingredients, excipients and medicines (cf. European Pharmacopoeia, 6.0, Volume 1, chapter 5.4, pp. 647-657).

Gamma-butyrolactone (GBL) has the remarkable property of also being a good solvent for salicylic acid and for benzoic acid. The fungistatic action of benzoic acid may be combined with the keratolytic action of the salicylic acid. The latter's keratolytic action facilitates the penetration of the antifungal after the abrasion, filing or grinding of the affected nail.

The association of benzoic acid and salicylic acid is known in the Whitfield ointment used in the treatment of mycosis intertrigo (cf. Goodman and Gilman, DORVAULT).

Benzoic acid and salicylic acid may advantageously be associated with the major antifungals as a solution in GBL, in antifungal preparations intended for the topical treatment of onychomycosis: they reinforce the action of such preparations and improve their diffusion into the damaged parts of the nail. Their concentration is advantageously of between 1 and 6% by weight of salicylic acid or benzoic acid or of each of these acids jointly introduced into the preparations.

Another advantage of gamma-butyrolactone (GBL) is its compatibility with the incorporation of agents such as colloidal silica or polyethylene glycols (PEG) which enable solutions of variable viscosity or gelled solutions to be prepared, or with emulsifying and detergent agents such as polyoxyethylenic alcohols (e.g. Cetomacrogol 1000) or Polysorbate 80. These agents are advantageously incorporated in proportions of between 1 and 20% for the PEG and 1 and 5% by weight for the colloidal silica and emulsifying and detergent agents.

Viscous and gelled solutions in GBL are advantageously prepared as, with regard to the subungual and periungual areas, these ensure better impregnation of the area to be treated, thus, better penetration of the active ingredients (keratolytic and anti-fungal) and, consequently, better therapeutic effectiveness.

Furthermore, filmogenic agents, such as acrylate copolymer and methyl acrylate (EUDRAGIT) or copolymer of methyl-vinyl-ether and maleic acid monobutyl ester may be advantageously incorporated in proportions of between 0.5 and 10% by weight.

The preparation may or may not be tinted using a colorant which may be of mineral or organic origin, but more advantageously vegetal.

GBL additionally has the advantageous property of being miscible in water or with organic solvents such as benzyl alcohol, ethanol, propylene glycol, acetone, ethyl acetate, butyl acetate or dimethylsulphoxide (DMSO).

GBL may be used in combination with one or several of these organic solvents, in proportions of between 5 and 90% by weight.

The following example preparations illustrate various compositions according to the invention.

Example 1: Simple Solution (I)

| | | |
|---|---|---|
| Salicylic acid | 3 | g |
| Benzoic acid | 3 | g |
| Propylene glycol | 10 | g |
| Gamma-butyrolactone | 84 | g |
| TOTAL | 100 | g |

Example 2: Viscous simple solution (II)

| | | |
|---|---|---|
| Salicylic acid | 3 | g |
| Benzoic acid | 3 | g |
| Propylene glycol | 10 | g |
| Gamma-butyrolactone | 82 | g |
| Colloidal silica | 2 | g |
| TOTAL | 100 | g |

Example 3: Gelled simple solution (III)

| | | |
|---|---|---|
| Salicylic acid | 3 | g |
| Benzoic acid | 3 | g |
| Propylene glycol | 10 | g |
| Gamma-butyrolactone | 81 | g |
| Colloidal silica | 3 | g |
| TOTAL | 100 | g |

Example 4: Filmogenic simple solution (IV)

| Salicylic acid | 3 | g |
|---|---|---|
| Benzoic acid | 3 | g |
| Propylene glycol | 10 | g |
| Gamma-butyrolactone | 81 | g |
| Eudragit RL 100 | 4 | g |
| Colorant | qs | |
| TOTAL | 100 | g |

On the basis of one or other of the previous antifungal compositions, preparations may be obtained containing, in addition, a major antifungal such as griseofulvin, or an imidazole derivative (for example, bifonazole, Econazole) or ciclopirox (or ciclopirox olamine), or amorolfine, or terbinafine, in proportions advantageously of between 0.5 and 10% by weight.

The following example preparations are griseofulvin-based.

Example 5: Griseo solution (Griseo I)

| | | |
|---|---|---|
| Griseofulvin | 4 | g |
| Salicylic acid | 2 | g |
| Benzoic acid | 2 | g |
| Propylene glycol | 10 | g |
| Gamma-butyrolactone | 82 | g |
| TOTAL | 100 | g |

Example 6: Viscous Griseo solution (Griseo II)

| | | |
|---|---|---|
| Griseofulvin | 4 | g |
| Salicylic acid | 2 | g |
| Benzoic acid | 2 | g |
| Propylene glycol | 10 | g |
| Gamma-butyrolactone | 80 | g |
| Colloidal silica | 2 | g |
| TOTAL | 100 | g |

Example 7: Gelled Griseo solution (Griseo III)

| | | |
|---|---|---|
| Griseofulvin | 4 | g |
| Salicylic acid | 2 | g |
| Benzoic acid | 2 | g |
| Propylene glycol | 10 | g |
| Gamma-butyrolactone | 79 | g |
| Colloidal silica | 3 | g |
| TOTAL | 100 | g |

Example 8: Filmogenic Griseo solution (Griseo IV)

| Griseofulvin | 4 | g |
|---|---|---|
| Salicylic acid | 2 | g |
| Benzoic acid | 2 | g |
| Propylene glycol | 10 | g |
| Gamma-butyrolactone | 79 | g |
| Eudragit RL 100 | 4 | g |
| Colorant | qs | |
| TOTAL | 100 | g |

In addition to its property as a solvent, GBL has the remarkable advantage of possessing good biological tolerance. The LD50, for the rat, by mouth, is of 17.2 ml/kg (SMYTH et coll., 1969). The SOS Chromatest and Mutatest show no genotoxic reaction (QUILLARDET et coll., 1985); there is no alteration of DNA (HOY et coll., 1984, KEMMINKI K., 1981); the Mutascreen Test shows no mutagenic action (FLACK et coll., 1985).

Its tolerance in subungual and periungual application is good and perfectly compatible with the desired therapeutic effect. In an abraded nail, GBL enables an excellent penetration of the active ingredients, which is reinforced by the keratolytic action of the salicylic acid. However, pure or concentrated GBL is irritating to the eyes and the mucous membranes and must be used with care so as to avoid contact with these sensitive areas.

GBL's good biological tolerance is essentially linked to its property of being a precursor, by simple hydrolysis, of 4-hydroxybutyric acid which is a normal constituent of the human organism (MUYARD et LABORIT, 1977; MAMELAK, 1989). 4-hydroxybutyric acid sodium salt is the active substance of a proprietary medical product used intravenously.

GBL itself has been used as a light hypnotic in the form of a syrup, at does by mouth of between 50 and 100 mg/kg (LANSADE et coll., 1985).

Antifungal preparations for topical use as a solution in GBL, as described previously, as particularly suited to the treatment of nail mycosis (onychomycosis).

For the nails of the foot, the activity of these preparations can be observed after 3 to 4 months of weekly or bi-weekly treatment, after which time the healthy nail begins to grow appreciably.

If the nail matrix is not completely damaged, the preparations described here do not require an associated systemic treatment, on condition that the diseased parts of the nail are carefully abraded by filing and/or grinding, and that measures of strict hygiene are followed for the hands and feet.

1. An antifungal preparation comprising, in solution, gamma-butyrolactone as a solvent vehicle.

2. The antifungal preparation according to claim 1, further comprising between 1 and 6% by weight of salicylic acid.

3. The antifungal preparation according to claim 1, further comprising between 1 and 6% by weight of salicylic acid and between 1 and 6% by weight of benzoic acid.

4. The antifungal preparation according to claim 2, further comprising between 0.5 and 7% by weight of griseofulvin.

5. The antifungal preparation according to claim 1, further comprising between 0.5 and 5% by weight of an antifungal from the group of imidazoles.

6. The antifungal preparation according to claim 1, further comprising between 0.5 and 10% by weight of ciclopirox or ciclopirox olamine.

7. The antifungal preparation according to claim 1, further comprising between 0.5 and 10% by weight of amorolfine.

8. The antifungal preparation according to claim 1, further comprising between 0.5 and 5% by weight of terbinafine.

9. The antifungal preparation according to claim 1, further comprising between 5 and 50% by weight of one or several biologically acceptable solvents.

10. The antifungal preparation according to claim 1, further comprising between 1 and 10% of an agent enabling a viscous or gelled solution to be obtained.

11. The antifungal preparation according to claim 1, further comprising between 0.5 and 10% by weight of a filmogenic agent.

12. The antifungal preparation according to claim 11, further comprising an emulsifying and detergent agent.

13. The antifungal preparation according to claim 1, further comprising a tinting agent selected from the group consisting of mineral, organic, and vegetal colorants.

14. A method for treating onychomycosis, comprising topical application of the antifungal preparation of claim 1 to the nail.

15. The antifungal preparation according to claim 9, wherein the one or several biologically acceptable solvents are selected from the group consisting of ethanol, propylene glycol, acetone, ethyl acetate, butyl acetate, and dimethylsulphoxide.
